(19) United States
(12) Patent Application Publication
(10) Pub. No.: US 2021/0068388 A1
(43) Pub. Date: Mar. 11, 2021

(54) A MULTIPURPOSE POTENTIATOR COMPOSITION AND THE METHODS THEREOF

(71) Applicant: T. Umakanthan, Tamil Nadu (IN)

(72) Inventors: T. Umakanthan, Tamil Nadu (IN); U. Umadevi, Tamil Nadu (IN)

(21) Appl. No.: 16/770,657

(22) PCT Filed: Nov. 21, 2018
(86) PCT No.: PCT/IN2018/050768

§ 371 Date: Jun. 8, 2020

(30) Foreign Application Priority Data
Dec. 7, 2017 (IN) 201741043843

(51) Int. Cl. A01N 1/02; A01N 3/00; A61K 47/02; A01N 59/08; A01N 59/00; A01N 47/06; A61K 39/39; C10L 10/00; C08J 7/06; C04B 41/45; C04B 41/50; D06M 11/76; D06M 11/64; D06M 11/13; A23L 3/358; B05D 1/02; A24B 15/28

(52) U.S. Cl. CPC A01N 1/021; D06M 2101/06; A61K 47/02; A01N 59/08; A01N 59/00; A01N 47/06; A61K 39/39; C10L 10/00; C08J 7/06; C04B 41/4543; C04B 41/5009; C04B 41/501; D06M 11/76; D06M 11/64; D06M 11/13; A23L 3/358; B05D 1/02; A24B 15/287; C08J 2321/00; A23V 2002/00; A01N 3/00

(57) ABSTRACT

Disclosed is a multipurpose potentiator composition comprising: sodium carbonate monohydrate, sodium carbonate anhydrous, potassium nitrate, sodium chloride and water such that the potentiating composition is applied to alter physical or chemical properties or both of a substance on which the potentiating composition is applied. Also provided is a container for holding the composition.

### FIELD OF INVENTION

The present invention is directed to a multipurpose composition and more particularly to a composition that resides in a container to potentiate safely and economically the natural potency of a substance made of atoms.

### BACKGROUND OF INVENTION

Delivering compositions incorporating a combination of viable preparations for enhancing the efficacy of various active substances in variety of applications, including but not limited to food, petrochemical, agro chemical, rubber, textile, cement, etc have been known by those skilled in art. However, indiscriminate spraying of such potentiated compositions has caused their relentless administration and delivery, which has adversely impacted their overall acceptability in midst of stringent environmental conservation measures being adopted worldwide.

Such dosage forms, thus, have invariably impacted their admittance in various industries like agrochemicals, pharmaceuticals, food and so on so forth. The release rate and amount of composition to be administered has been of considerable significance in recent years where the coherent efforts are being made to intensify the effect of potentiated composition without considerably enhancing the quantity of dosage. None of the currently existing technologies provides a single, economical, safe, easy and harmless way for adequate dispensation of these solutions to provide for desired efficacy and potency.

Furthermore, the existing spray potentiators are not guided on how to be used for their effective application. The essentials of spraying the appropriate amount of composition which is also dependent on various other combination of factors vis-à-vis orifice diameter of container, distance from which the solution shall be suitably sprayed, pressure of ejection, time of spray etc, are required to be monitored with further precision.

Last, but not the least the bond parameters of the usable upon which the composition is sprayed, are presently altered by conventional ways of heating, cooling, drying, chemical addition, mechanical, electrical or use of radio energies. This process is laborious, cumbersome, costly, mostly industrial based which requires huge amount of energy.

In order to overcome aforementioned limitations, there exists a need for a single, cost effective universal spray potentiator that can alter the physical and chemical characters of daily usable items by spraying the regulated amount of universally applicable potentiator composition under guided conditions thereby obviating the need for energy requirements.

### OBJECT OF THE INVENTION

The primary objective of the present disclosure is to provide a single, economical and safe multipurpose potentiating composition to enhance the efficacy of the potentiating composition.

In one other objective of the present disclosure, a cost effective, universally applicable spray potentiator composition capable of achieving desired efficacy in regulated amount, is proposed.

Another objective of the present disclosure is to provide an enhanced nutritive value, taste, aroma and shelf life to agro products and food compositions without requiring any further addition of preservatives and chemicals.

Another objective of this disclosure is to potentiate the drugs and pharmaceutical compositions with increased shelf life, minimized adverse side effects, reduced intake of drug dosage, thereby host stress reduced to consumers.

Yet another objective of the disclosure is to provide a single, unique and universal therapy to cure any of human, animals or plant diseases with ease and in optimized way using universal spray potentiator equipped to spray the viable potentiator composition.

Yet other objective of the present disclosure is to facilitate potentiation of vaccine thereby increasing antibody production or reducing the vaccine dose without diluting the amount of antibody made available to the diseased body. Thereby host stress and production cost reduced and economy saved.

It is another object of the present disclosure to provide for reduced consumption of petrochemicals to contribute to reduction in environmental pollution, and for combination with allied products like grease, lubricants, coolant oils, petroleum by products etc. to improve upon their quality.

It is a further object of the present disclosure to enable repeated use of edible oils up to almost four times while eliminating causes of cardiac diseases or other health hazards caused by repeated heating of edible oils.

It is another object of the present disclosure to increase the burning oil time of oils such as kerosene, gingelly, and ghee thus promising their usage in traditional lamps in an even more economical way.

One other object of the present disclosure contributes to increase in tensile strength of materials like rubber, polyester yarns, and concrete compressive strength of cement etc., and thence making their applicability more purposeful with reduced amount of raw material without having to compromise on quality of end product.

In yet another object of the present disclosure, anti radiation reduction up to 60% is achievable from electrical and electronic components that otherwise causes serious impact on health of users.

In still other object of the present disclosure, a spray potentiator composition capable of neutralizing toxins naturally present in tobacco and its by products, and alcohol without compromising on its satiety value, is provided.

Another significant object of the present disclosure is to provide a sprayable potentiating composition capable of reducing the agrochemical quantity requirement and contributing to increased yield of agro crops while the soil fertility levels remain intact.

These and other objects will become apparent from the ensuing description of the present invention.

### SUMMARY OF THE INVENTION

The present invention is directed to a spray potentiator comprising a container containing a potentiating composition that includes a combination of sodium carbonate monohydrate, sodium carbonate anhydrous, potassium nitrate, sodium chloride and water in proportions such that the potentiating composition is sprayed in a regulated amount at a determined pressure to alter physical or chemical properties or both of a usable upon or externally over which the potentiating composition is sprayed.

In another preferred embodiment, the sodium carbonate monohydrate constitutes 35%, the anhydrous sodium carbonate constitutes 30%, the potassium nitrate constitutes 20%, and the sodium chloride constitutes 15% of the potentiating composition.

These and other aspects, features and advantages of the present invention will be described or become apparent from the following detailed description of preferred embodiments.

### BRIEF DESCRIPTION OF THE ACCOMPANYING DRAWINGS

FIG. 1 illustrates a container for spraying the potentiating composition wherein

FIG. 1(a) is a frontal view; FIG. 1(b) is side view, and FIG. 1(c) is longitudinal section view in accordance with the present invention;

FIG. 2 illustrates changing the sweetness where FIG. 2a is the fluorescence spectra of samples; 2(b) is the PXRD spectra of the samples; and FIG. 2c is the FT-IR spectra of the sample in accordance with the present invention;

FIG. 3 is an image of bacterial killing effect in accordance with the present invention;

FIG. 4 is the image of patients before and after treatment in accordance with the present invention;

FIG. 5 is the image of emission trial reports wherein a) before the spray and b) after the spray in accordance with the present invention;

FIG. 6 is the FTIR spectra of the cement a) Control (Non-spray) and b) trial (sprayed) in accordance with the present invention;

FIG. 7 is the PXRD spectra of the cement a) Control (before the treatment) and b) sprayed form (after the treatment) in accordance with the present invention;

FIG. 8 is the FTIR spectra of the tobacco powder a) Control (Non-spray) and b) trial (sprayed) in accordance with the present invention;

FIG. 9 is the PXRD spectra of the tobacco powder a) Control (Non-spray) and b) trial (sprayed) in accordance with the present invention;

FIG. 10 is the image of the plant in accordance with the present invention; &

### DETAILED DESCRIPTION OF THE INVENTION

It has to be understood and acknowledged for this specification and claims, that the term “spray potentiator” refers to a composition which could alter the physical properties or chemical properties or both of a substance upon which the composition is applied in a safe, economical, easy and harmless manner. The term “spray potentiator”, “universal potentiator”, “multipurpose spray potentiator”, “universal spray potentiator”, “spray potentiator composition” is used herein interchangeably and the phrases herein define the composition which is made up of a specific combination of sodium carbonate monohydrate, anhydrous sodium carbonate, potassium nitrate and sodium chloride. Before the present “universal spray potentiator” is described, it is to be understood that this disclosure is not limited to a particular form. It is also to be understood that this disclosure is not limited to the particular device for spraying potentiated composition, as described, since it may vary within the specification indicated to meet varying requirements in different fields of application.

It is also to be understood that the terminology used in the description is for the purpose of describing the particular versions or embodiments only, and is not intended to limit the scope of the present invention, which will be limited only by the appended claims. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. The disclosed embodiments are merely exemplary methods of the invention, which may be embodied in various forms.

In accordance with the first embodiment, a multi-purpose, economical, safe, easy and harmless spray potentiator composition is disclosed. Primarily, the spray potentiator is configured to potentiate the natural potency of basic usable substrate like food, agro chemicals, petrochemicals, rubber, textile, cement etc. such that the quantity of basic usable commodity is reduced, the natural resources are conserved thereby economizing the ways to enhance the intensity of active ingredient and concurrently prevent indiscriminate polluting of environment.

The substance according to present invention is selected from a group consisting of lady finger, pumpkin, carrot, beans, brinjal, bottle guard, bitter guard, jamun, banana, strawberry, mango, papaya, chicken gravy, mutton gravy, raw meat, boiled egg, half boiled egg, omlette, raw egg, raw milk, boiled milk, market raw milk, market boiled milk curd, butter milk, butter, clarified butter, jasmine, rose, chrysanthemum, moringa leaves, spinach, antibiotic, human transfusion blood, food and mouth disease vaccine, petrol, diseal, liquefied petroleum gas, kerosene, gingelly oil, rubber material, cotton yarn, cement, electric and electronic equipments, tobacco products or alcohol. All the substance as used in the present invention is available in any local market.

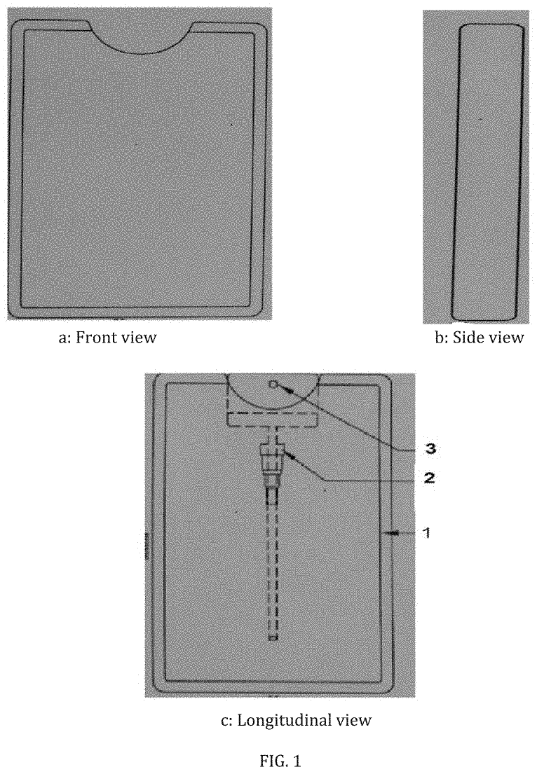

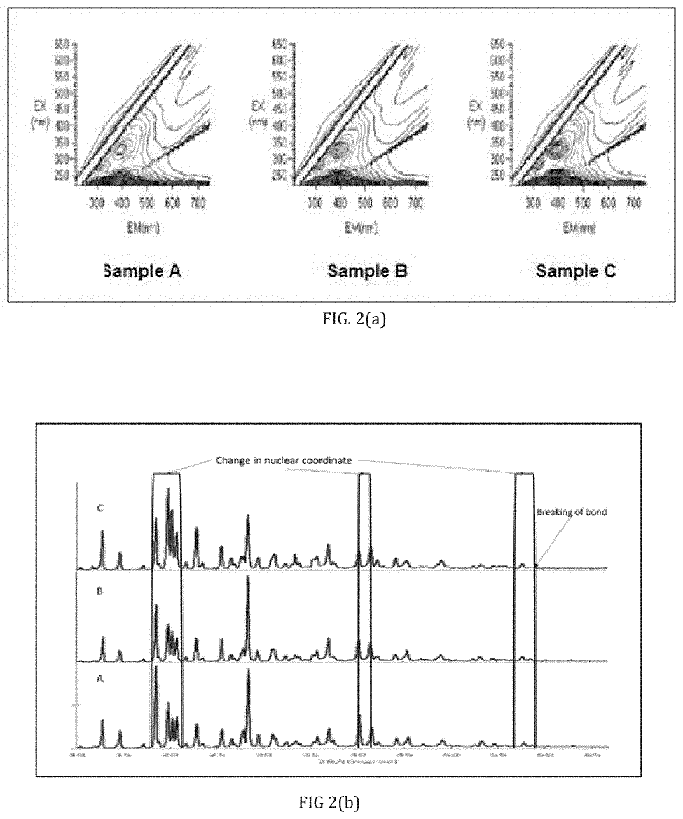

Accordingly, in accordance with one preferred aspect of the present disclosure, a universal spray potentiator comprises of a container (1) that holds the viable potentiating composition. The container, for example, can be made of a thermoplastic polymer, such as polypropylene plastic having dimensions in range of 86×55×11 mm and the said container includes a plunger (2) and an orifice (3). The potentiator is designed to spray the potentiated composition through an orifice (3) having a diameter ranging between 0.3 mm to 0.4 mm, and preferably 0.375 mm (FIG. 1).

In one preferred embodiment, while the potentiated composition is sprayed from the orifice of the container, a pressure of no less than 3900 Kpa is applied, which is averaged over a 0.2 second time period of a spray ejection. The amount of potentiated composition ejected per spray shall vary between 60-70 micro liters, preferably 62 micro liters with a permissible standard deviation of 5 micro liters. The non-volatile material in the sprayed composition amounts to 153 mg/ml, and almost one micro gram of weight loss from the ejected material occurs immediately upon spraying. In order to achieve the desired result, according to present invention, maximum 4 times spray/day to that substance, is required.

In one significant aspect of the present disclosure, the potentiating composition comprises of a combination of sodium carbonate monohydrate, sodium carbonate anhydrous, potassium nitrate, sodium chloride and water. In one exemplary embodiment, the potentiating composition comprises approximately 35% of sodium carbonate monohydrate, 30% of sodium carbonate anhydrous, 20% of potassium nitrate, 15% of sodium chloride and sufficient quantity of water, which makes the composition capable of being sprayed effectively from a determined distance to achieve desired potentiating effects.

In one preferred embodiment, when the potentiating composition is sprayed upon the usable commodity, the ions are ejected along with water mist, oscillate among themselves and generate Electro Magnetic waves (low energy photons). The potentiating composition is preferably sprayed from a distance of at least 6-12 inches preferably 9-12 inches over and above the packets containing basic usable like food, blood, tobacco, alcohol, drugs, vaccines, cement, liquid and gaseous fuels, agrochemicals, etc, (including therapy for diseases).

In one embodiment, the packets enclosing the basic usable may be of any type such as polythene, wood, paper, glass, metal, etc, (including human/animal skin). Energy is generated while spraying the potentiated composition that passes through the packet to activate the content held inside the usable packet that causes alterations in bond parameters, including relaxing or tightening or breaking or formation of new bond. Once the bond parameters are affected, the physical and chemical characters of the usable are also spontaneously altered, leading to enhancement of natural characters of the content inside the packet depending on change in degree of bond parameters. Following the activation of packet content, the energy passes away through the other side of the packet.

The sprayed ionizing energy does not possess the ability to make any intervening media radioactive, and also does not get stored while passing there through. The process of interaction of such energy with the usable inside the packet to activate the contents of the usable occurs among magnetic fields and waves, which makes the results of bond alteration quickly. Further, since the composition is sprayed from a safe distance of almost 6-12 inches, the potentiator is highly economical, without adverse effect, safe for use, and hence can be used by anybody without requiring specialized training and skills.

In an embodiment of present disclosure, the universal spray potentiator finds its application in food industry to increase the taste, aroma, shelf life of agro products and food including vegetarian and non vegetarian food without requiring addition of any preservative or additive chemicals. Even if there exists a need to add any additional preservatives, additive and chemical, these residues get denatured during potentiation processing. The use of potentiating composition not only enhances the nutritive value of food, but also increases its shelf life from 6 months to up to 3 years, besides contributing to enhanced taste and aroma of food.

In an embodiment of present disclosure, this single and unique therapy is conceptualized to cure any animal or plant diseases, and human diseases including all diseases related to skin, nails, alimentary, endocrine, ear, nose, throat, cardiovascular, immune, musculoskeletal, CNS and psychic, reproductive, respiratory, urinary etc.

The present invention is now illustrated by the way of non-limiting examples:

### Example 1: Effect of Spray Potentiator Composition on Food Products

A solution is prepared by adding 35% of sodium carbonate monohydrate, 30% of sodium carbonate anhydrous, 20% of potassium nitrate, 15% of sodium chloride in water and the solution was placed inside a container (polypropylene plastic made having dimensions in range of 86×55×11 mm, capacity 20 mL) as described in FIG. 1. All the chemicals as above were purchased from I.L.E.CO., NO. 19, Thalavai street, Madurai-625 001, Tamil Nadu, India. A trial was conducted by spraying the composition once or twice to raw vegetables, fruits, meat, egg products, milk and milk products depending upon the weight of the substance, wherein the concentration of the said solution is 153 mg/ml and pH of the said solution is 10.30-12.13; pressure at the time of pressing is 3900 KPa and the quantity of the solution delivered to these substrate is 0.06 mL.

The results are tabulated in Table 1 to 5 for example, the sprayed vegetables have shown a marked improvement in their extended unspoiled stage under both room temperatures and refrigerated conditions in Table 1 likewise, in case of fruits, there has been an extension of almost 4-10 times in number of days in which the fruits can be kept under unspoiled state when experimented under both room and refrigerated temperatures, as given in Table 2.

TABLE 1
Trial with vegetables

| | | Room temperature | | Refrigerated condition | |
|---|---|---|---|---|---|
| | | (27° C.) | | (4° C.) | |
| | | No. of days remained | | No. of days remained | |
| | | unspoiled | | unspoiled | |
| | | | Trial | | Trial |
| | | | (Sprayed) | | (Sprayed) |
| SI. | Samples | Control (Non | (Inventive | Control (Non | (Inventive |
| no | used | sprayed) | example) | sprayed) | example) |
| 1 | Lady's finger | 6 | 24 | 7 | 42 |
| 2 | Pumpkin | 2 | 12 | 4 | 20 |
| 3 | Carrot | 3 | 30 | 5 | 38 |
| 4 | Beans | 5 | 15 | 7 | 22 |
| 5 | Brinjal | 3 | 15 | 5 | 29 |
| 6 | Bottle guard | 3 | 25 | 5 | 37 |
| | (Home | | | | |
| | cultivated) | | | | |
| 7 | Bitter guard | 3 | 13 | 5 | 17 |
| | (Home | | | | |
| | cultivated) | | | | |

wherein,
the ejection volume is 0.06 mL/spray;
the potentiating composition is sprayed at 3900 Kpa;
the pH of the solution is 10.30-12.13;

TABLE 2
Trial with Fruits

| | | Room temperature (27° C.) | | | |
|---|---|---|---|---|---|
| | | No. of days remained | | Refrigerated condition (4° C.) | |
| | | unspoiled | | No. of days remained | |
| | | | Trial | unspoiled | |
| | | | (Sprayed) | | Trial (Sprayed) |
| SI. | Samples | Control (Non | (inventive | Control (Non | (inventive |
| no | used | sprayed) | example) | sprayed) | example) |
| 1 | Jamun | 2 | 8 | 5 | 9 |
| 2 | Banana | 4 | 12 | 6 | 27 |
| 3 | Straw- | 2 | 20 | 5 | 32 |
| | berry | | | | |
| 4 | Mango | 3 | 20 | 5 | 37 |
| 5 | Papaya | 2 | 19 | 4 | 33 |

### Wherein,

the ejection volume is 0.06 mL/spray;
the potentiating composition is sprayed at 3900 Kpa;
the pH of the solution is 10.30-12.13.

Next, as shown in Table 3 in case of sprayed meat and egg based products, the prepared gravies or end preparations can be stored for as long as 300 days when kept under refrigerated conditions, while it can be stored for almost a week when kept under room temperatures. With dairy and dairy based products, as shown in Table 4 the sprayed products have shown an extended shelf life of up to 80-90 days under refrigerated conditions and 10-15 days under room temperatures, which is almost 5-7 times more than the shelf life under non sprayed conditions.

TABLE 3
Trial on meat and egg products

| | | Room temperature | | Refrigerated condition | |
|---|---|---|---|---|---|
| | | (27° C.) | | (4° C.) | |
| | | No. of days remained | | No. of days remained | |
| | | unspoiled | | unspoiled | |
| | | | Trial | | Trial |
| | | | (Sprayed) | | (Sprayed) |
| SI. | Samples | Control (Non | (inventive | Control (Non | (inventive |
| no | used | sprayed) | example) | sprayed) | example) |
| 1 | Chicken | 1 | 5 | 3 | 380 |
| | gravy | | | | |
| 2 | Mutton | 1 | 3 | 3 | 45 |
| | gravy | | | | |
| 3 | Raw meat | 1 | 4 | 4 | 275 |
| 4 | Boiled egg | 2 | 5 | 5 | 45 |
| 5 | Half boiled | 1 | 7 | 4 | 275 |
| | egg | | | | |
| 6 | Omlette | 2 | 8 | 5 | 380 |
| 7 | Raw egg | 3 | 6 | 6 | 361 |

wherein,
the ejection volume is 0.06 mL/spray;
the potentiating composition is sprayed at 3900 Kpa;
the pH of the solution is 10.30-12.13.

TABLE 4
Trial on meat and egg products

| | | Room temperature | | Refrigerated condition | |
|---|---|---|---|---|---|
| | | (27° C.) | | (4° C.) | |
| | | No. of days remained | | No. of days remained | |
| | | unspoiled | | unspoiled | |
| | | | Trial | | Trial |
| | | | (Sprayed) | Control | (Sprayed) |
| SI. | | Control (Non | (inventive | (Non | (inventive |
| no | Samples used | sprayed) | example) | sprayed) | example) |
| 1 | Raw milk | 1 | 3 | 2 | 38 |
| 2 | Boiled milk | 1 | 4 | 2 | 46 |
| 3 | Market raw | 1 | 5 | 2 | 47 |
| | milk | | | | |
| 4 | Market boiled | 1 | 6 | 3 | 51 |
| | milk | | | | |
| 5 | Curd | 1 | 7 | 4 | 75 |
| 6 | Butter milk | 2 | 11 | 4 | 80 |
| 7 | Butter | 3 | 9 | 7 | 46 |
| 8 | clarified butter | 4 | 15 | 10 | 94 |

wherein,
the ejection volume is 0.06 mL/spray;
the potentiating composition is sprayed at 3900 Kpa;
the pH of the solution is 10.30-12.13

With flowers and leafy products like jasmine, rose, spinach etc, it has been witnessed in Table 5 that the sprayed products has an extended life of up to 6-8 days under refrigerated conditions, and up to 5 days under room temperature conditions. Even the food grains and the pulses can be preserved by spraying the potentiated composition on them and storing them in bags. This has led to increase by up to 100 times than normal in shelf life of grains and pulses.

TABLE 5
Trial with flowers and leafy vegetable

| | | Room temperature (27° C.) | | Refrigerated | |
|---|---|---|---|---|---|
| | | No. of days remained | | condition (4° C.) | |
| | | unspoiled | | No. of days remained | |
| | | | Trial | unspoiled | |
| | | Control | (Sprayed) | Control | Trial (Sprayed) |
| SI. | | (Non | (inventive | (Non | (inventive |
| no | Samples used | sprayed) | example) | sprayed) | example) |
| 1 | Jasmine | 1 | 3 | 2 | 4 |
| 2 | Rose | 1 | 4 | 2 | 6 |
| 3 | Chrysanthe- | 2 | 5 | 5 | 7 |
| | mum | | | | |
| 4 | Moringa | 1 | 3 | 5 | 8 |
| | leaves | | | | |
| 5 | Spinach | 1 | 3 | 4 | 7 |

wherein,
the ejection volume is 0.06 mL/spray;
the potentiating composition is sprayed at 3900 Kpa;
the pH of the solution is 10.30-12.13

In respect of non cooked food products, say for example, coconut and gram chutney, which usually gets spoiled in 6-12 hours even if refrigerated, here after spraying the potentiated composition the chutney start to diminish in taste and flavor only after 72 hours. In fact under the refrigerated conditions, the sprayed foods found to be more palatable and colorful up to 3-6 months.

Trail on glucose powder for increasing or decreasing the sweetness

This trial was conducted on glucose powder at Department of Chemistry, Tokyo University of Science, Tokyo, Japan.

Sample-A: Glucose powder (untreated);
Sample-B: Glucose powder upon which the potentiating composition was sprayed two times (treated); &
Sample-C: Glucose powder upon which the potentiating composition was sprayed six times (Extremely treated).

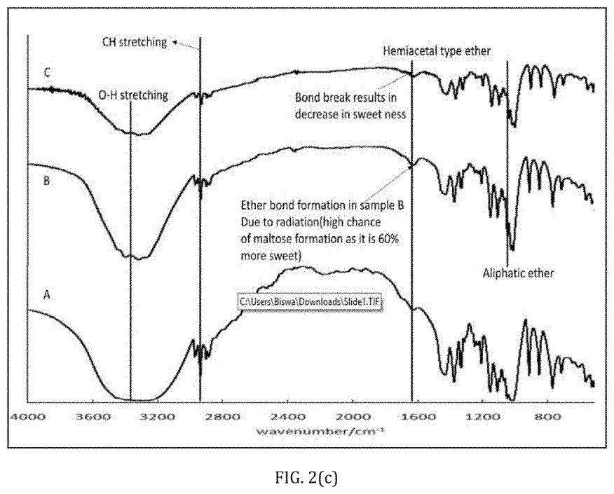

As shown in FIG. 2(a), there is three dimensional fluorescence spectra of sample-A, sample-B and sample-C wherein the excitation peak of the sample-B and sample-A is at 330 nm and the emission peak is at 380 nm. The increased fluorescence intensity in the sample B may be because of the changes in the structure and environment of the glucose. The rapid decrease in fluorescence intensity of the sample-C may be due to few breaks of the chemical bonds in the glucose.

As shown in FIG. 2(b), there is three PXRD of sample-A, sample-B and sample-C wherein the changes in the peak intensities at 200 nm and 400 nm may be because of the changes in nuclear coordinates. The changes in the peak intensity at 580 nm may be because of the chemical bond breaking in the sample C.

As shown in FIG. 2(c), there is three FT-IR spectra of sample-A, sample-B & sample-C. wherein ether bond is stretching near 1600 cm⁻¹ in sample B which implicates the formation of maltose from two glucose molecules. However, the same bond disappears in sample C implicating the ether bond breakage because of extreme radurization of glucose powder. The difference in CH-stretching (at 2950 cm⁻¹) and OH-stretching (at 3300 cm⁻¹) in the sample B corresponding to untreated sample implicates the corresponding changes in the CH and OH environment because of the ether bond formation in that vicinity. The formation of ether bond between two molecules of glucose is again supported from the changes in the ether bond stretching at 1050 cm⁻¹.

Findings:
i) Sample-B was found sweet than sample-A
ii) Sample-C was found bitter than sample-A.

### Example 2: Effect of Spray Potentiator Composition on Drugs

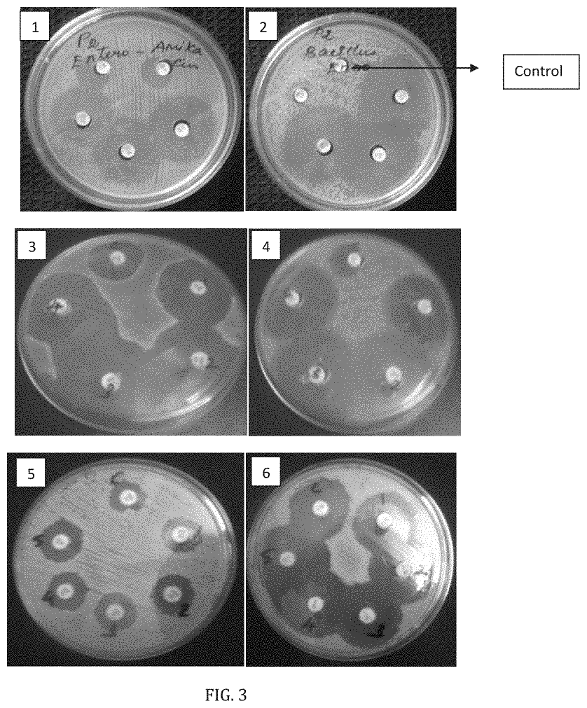

The composition, container and spraying parameter were followed as described in Example 1. The composition is sprayed finds its practical application in medical industry—drugs, pharmaceuticals and cosmetics. Upon conducting a trial (anti-bacterial effect of the composition) at Veterinary hospital, Kanjampatty, Pollachi, Tamil Nadu, India it was found (FIG. 3) that spraying the composition, the antibiotic gets potentiated which does not necessitate any additional quantity of antibiotic to be supplemented for achieving same level of natural potency. There has been an increase of potency (bacterial killing effect) to the tune of 3-153% which has made the process extremely economical as same level of efficacy is achieved without increasing further the dosage amount. Also, the shelf life of the drug is enhanced, resistance to drug being reduced effectuating increased drug impact without any adverse reaction. Using Universal Potentiator spray a few number of antibiotics are enough for treatment for many thousands of years.

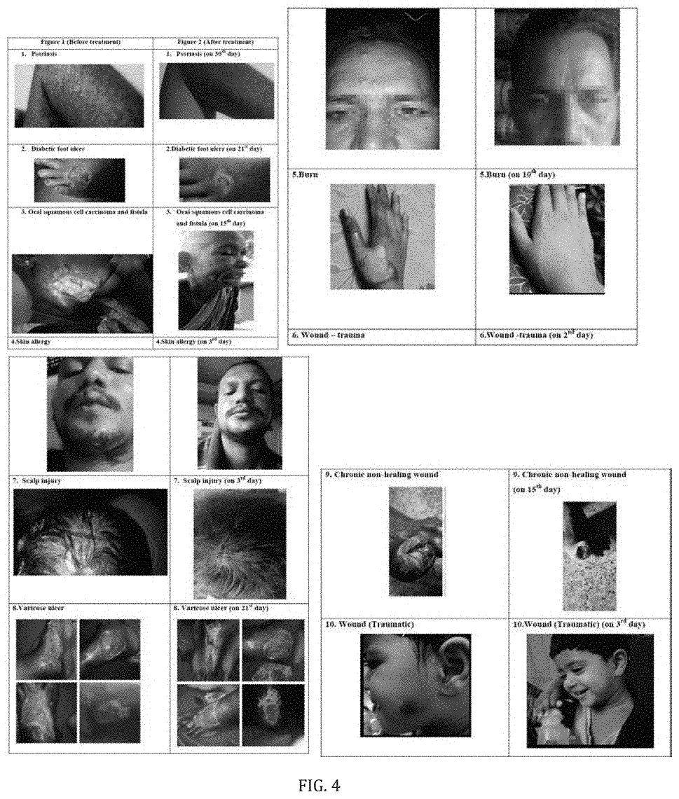

The present inventors observed the recovery (before and after treatment) of the patient (FIG. 4) within 30 days from the earliest day of the spraying on a disease selected from a group consisting of psoriasis, diabetic foot ulcer, oral squamous cell carcinoma and fistula, skin allergy, burn, wound-trauma, scalp injury, varicose ulcer, chronic non-healing wound, wound (traumatic) Generally, number of the spraying is one or two sprays twice per day.

### Example 3: Effect of Spray Potentiator on Human Transfusion Blood

The composition, container and the spraying parameter was followed as described in Example 1. The potentiating composition is observed to preserve the human transfusion blood for more than 5 weeks, thereby extending the shelf life up to 1-2 years. This has facilitated conservation of banked blood which otherwise is wasted away owing to its short life span of 4-6 weeks. Furthermore, potentiation of vaccine (both for use in human and veterinary medicines) can be increased which can further increase antibody production. Here, about 10-22% of increase in quantity of antibody producible is seen. In other words, 10-22% of quantity of vaccine to be administered can be reduced, thus reducing the stress on host as well.

### Example 4: Effect of Spray Potentiator Composition on Petrochemicals

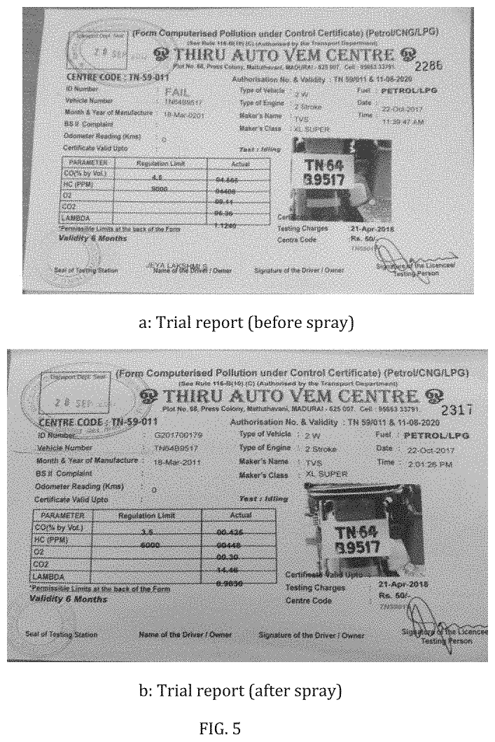

The composition, container and spraying parameter were followed as described in Example 1. In example 4, use of novel, single, universally applicable potentiator can be seen in petrochemical industry where the potentiating composition assists in reducing the consumption of petrochemical includes patrols, diesels, liquefied petroleum gas (LPG) up to 30% and simultaneously preventing environmental pollution contributed majorly by petrochemical industry. Specifically, the fuel consumption can be made to reduce by almost 12-59% with carbon oxide emissions being reduced by almost 98%, carbon dioxide emissions being cut down by almost 1-30%, nitrous oxide emissions up to 100% reduced while the oxygen levels has shown an increase by almost 0.5-82% in exhaust smoke. Emission trial report before and after the treatment is herewith enclosed (FIG. 5a and FIG. 5b). However, the above reduction in emission of various toxic gases is also dependent on various engine types. Such potentiator can also be combined to increase the quality of allied products like grease, lubricants, coolants, oils, petroleum by products.

### Example 5: Effect of Spray Potentiator Composition on Various Traditional Lamp Containing an Oil

The composition, container and spraying parameter were followed as described in Example 1. The spray potentiator is useful in reducing the health hazards arising out of consumption of repeatedly heated edible oils. With dispensing of potentiating composition, the oils can be kept at almost same consistency even after heating for 4 cooking cycles. This puts to rest all oil borne diseases while increasing the aroma, texture and shelf life of food prepared from potentiated edible oils.

Depending upon the weight of the substance, potentiating composition (0.5-1 wt %) is sprayed twice to the said substance and it was found that the potentiating composition has increased the burning time of various oils like by almost 158% for kerosene oil, 73% for gingelly oil and 23% of clarified butter. This can find a significant use in traditional lamps used in rural part of country as it will help the occupants economize their way of lighting homes.

TABLE 6
Burning time of the traditional lamp containing various oil

| Traditional lamp | Burning time |
|---|---|
| Control-kerosene oil | 53 minutes |
| (Comparative example) | |
| Kerosene oil + 1% potentiating composition | 2 hours 21 minutes |
| (Inventive example) | |
| Control-gingelly oil | 2 hours 13 minutes |
| (Comparative example) | |
| Gingelly oil + 1% potentiating composition | 3 hours 52 minutes |
| (Inventive example) | |
| Control- clarified butter (Comparative example) | 49 minutes |
| Clarified butter +1% potentiating composition | 57 minutes |
| (inventive example) | |

wherein,

the ejection volume is 0.06 mL/spray;

the potentiating composition is sprayed at 3900 Kpa;

the pH of the solution is 10.30-12.13

### Example 6: Effect of Spray Potentiator Composition on Rubber Materials

The composition, container and spraying parameter were followed as described in Example 1. The application of universal spray potentiator in material sciences is discussed. The trial was conducted at Sriram Karthic Polymers Pvt Ltd., Coimbatore, Tamil Nadu, India. Depending upon the weight of the rubber material, the potentiating composition is sprayed 3 times to a synthetic rubber. The potentiating composition (7.5 g) is sprayed 3 times to 1000 g of the rubber materials and the tensile strength is measured before and after addition of the potentiating composition. It was found that the tensile strength of rubber is increased by 43%. This has significantly reduced the quantity of rubber needed as a raw material in various industrial applications without having to compromise on any of life and strength of end product.

TABLE 7

| | SHORE | TS | |
|---|---|---|---|
| Sample No. | ‘A’ | (Kg/cm²) | EB % |
| 1 | 77 | 128 | 320 |
| 2 | 75 | 154 | 399 |
| 3 | 75 | 123 | 279 |
| 4 | 75 | 139 | 384 |
| 5 | 73 | 201 | 338 |
| 6 | 74 | 137 | 361 |
| 7 | 77 | 148 | 339 |
| 8 | 73 | 125 | 368 |
| 9 | 73 | 126 | 367 |
| 10 | 79 | 151 | 278 |
| 11 | 80 | 131 | 290 |
| 12 | 74 | 118 | 342 |
| 13 | 75 | 103 | 344 |
| 14 | 74 | 123 | 327 |
| 15 | 74 | 112 | 316 |

wherein,
the ejection volume is 0.06 mL/spray;
the potentiating composition is sprayed at 3900 Kpa;
the pH of the solution is 10.30-12.13

### Example 7: Effect of Potentiating Composition on Textile Products

In this trial, the mode of operation is socking.

The composition was as described in Example 1. The trial was conducted to confirm the effect of the present composition on textile product at Harshini textiles, Anaimalai, Coimbatore, Tamil Nadu, India where a cotton yarn was first soaked in 0.75% of universal potentiator solution for 5-10 minute and then the cotton yarn was dried under sun for 2 hours and then was subjected to measure various parameter as shown in Table 8. Comparison studies in Table 8 shows the strength in terms of CSP (count strength product) has been seen to increase by almost 16% even after washed with detergent. For example, the polyester yarn has an increase in strength by almost 42% and cotton yarn by 27-58% after being potentiated.

TABLE 8
Textile properties of the potentiator composition

| | Sample No. | | | | | | | |
|---|---|---|---|---|---|---|---|---|
| | 1 | 2 | 3 | 4 | 5 | 6 | 7 | 8 |
| | (Control) | (0.25%) | (0.5%) | (0.75%) | (1%) | (1.5%) | (1.75%) | (2%) |
| Count | 30.5 | 29.63 | 30.44 | 29.47 | 29.76 | 29.19 | 29.28 | 29.38 |
| CV % | 1.02 | 2.15 | 0.33 | 0.94 | 0.56 | 0.59 | 1.55 | 0.64 |
| Strength | 92.18 | 98.25 | 88.9 | 95.39 | 96.78 | 90.68 | 98.79 | 97.00 |
| CV % | 92.18 | 98.22 | 88.9 | 95.39 | 96.78 | 90.69 | 98.78 | 97.00 |
| CSP | 2790 | 2912 | 2709 | 2813 | 2882 | 2648 | 2893 | 2849 |
| U % | 9.60 | 9.16 | 10.84 | 10.37 | 11.23 | 11.85 | 11.92 | 11.26 |
| THIN −50 | 1 | 0 | 2 | 0 | 3 | 4 | 3 | 2 |
| THICK +50 | 23 | 25 | 49 | 46 | 93 | 99 | 82 | 74 |
| NEPS +200 | 55 | 39 | 77 | 73 | 109 | 85 | 86 | 61 |
| Total | 75 | 65 | 128 | 119 | 206 | 186 | 170 | 137 |
| Imperfections | | | | | | | | |
| Hairiness | 4.17 | 4.30 | 4.77 | 4.79 | 4.75 | 4.90 | 4.15 | 4.04 |

The standard evaluation criteria's is used to determine the samples which are i) the yarn should be with good CSP value; ii) The u % value should be lower and iii) it should have lower hairiness. According to Table 8, it is observed that Sample No. 02 is meeting with the criteria and thus is a good yarn.

### Example 8: Effect of Potentiator Composition on Concreate Materials

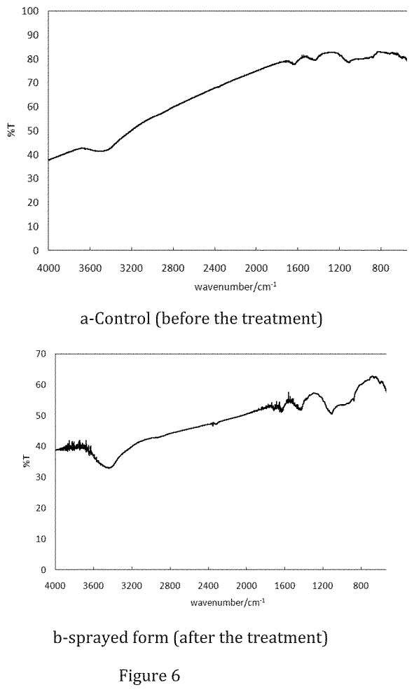

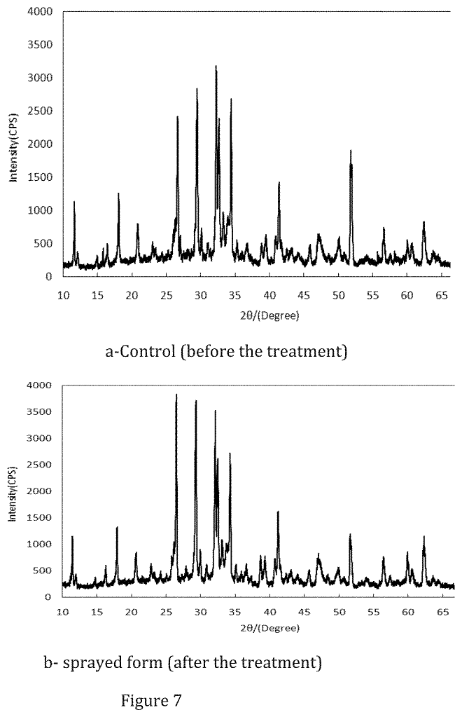

The composition, container and spraying parameter were followed as described in Example 1. The trial was conducted on cement at Department of Civil Engineering, Karpagam College of Engineering, Karpagam University, Coimbatore, Tamil Nadu, India. In this trial, the potentiating composition (7.5 g) is sprayed 2-4 times to 1000 g of cement and comprehensive strength of the concrete in N/mm² in different interval (7 days, 14 days and 28 days) was studied (Table 9-11) while spectroscopic analysis was conducted at Department of Chemistry, Tokyo University, Tokyo, Japan (FIGS. 6 and 7).

TABLE 9
Compressive Strength of Concrete In N/mm²
(7 Days Test Results)

| | | Compressive Strength of Concrete | | | |
|---|---|---|---|---|---|
| | Quantity of | N/mm² | | | Average Value |
| | the | Sample (1) - PFHM | | | of Compressive |
| SI. | potentiator | Specimen | Specimen | Specimen | Strength in |
| No | (%) | (1) | (2) | (3) | (N/mm²) |
| 1 | Control | 13.00 | 14.50 | 13.50 | 13.67 |
| | (comparative | | | | |
| | example) | | | | |
| 2 | 0.50% | 15.00 | 14.00 | 14.00 | 14.33 |
| | (inventive | | | | |
| | example) | | | | |
| 3 | 0.75% | 16.00 | 16.50 | 19.50 | 17.00 |
| | (inventive | | | | |
| | example) | | | | |
| 4 | 1.00% | 14.50 | 14.25 | 12.50 | 13.75 |
| | (inventive | | | | |
| | example) | | | | |

### Wherein,

the ejection volume is 0.06 mL/spray;
the potentiating composition is sprayed at 3900 Kpa;
the pH of the solution is 10.30-12.13

TABLE 10
Compressive Strength of Concrete in N/mm²
(14 Days Test Results)

| | | Compressive Strength of Concrete | | | Average |
|---|---|---|---|---|---|
| | Quantity of | N/mm² | | | Value of |
| | the | Sample (1) PFHM | | | Compressive |
| SI. | potentiator | Specimen | Specimen | Specimen | Strength in |
| No | (%) | (1) | (2) | (3) | (N/mm²) |
| 1 | Control | 18.00 | 18.00 | 18.50 | 18.17 |
| | (comparative | | | | |
| | example) | | | | |
| 2 | 0.50% | 19.25 | 20.50 | 18.25 | 19.16 |
| | (inventive | | | | |
| | example) | | | | |
| 3 | 0.75 | 25.00 | 25.50 | 25.50 | 25.33 |
| | (inventive | | | | |
| | example) | | | | |
| 4 | 1.00% | 20.50 | 21.75 | 20.50 | 20.91 |
| | (inventive | | | | |
| | example) | | | | |

### Wherein,

the ejection volume is 0.06 mL/spray;
the potentiating composition is sprayed at 3900 Kpa;
the pH of the solution is 10.30-12.13

TABLE 11
Compressive Strength of Concrete in N/mm2
(28 Days Test Results)

| | | Compressive Strength of Concrete | | | Average |
|---|---|---|---|---|---|
| | Quantity of | N/mm2 | | | Value of |
| | the | Sample (1) PFHM | | | Compressive |
| SI. | potentiator | Specimen | Specimen | Specimen | Strength in |
| No | added in (%) | (1) | (2) | (3) | (N/mm²) |
| 1 | Control | 21.00 | 21.00 | 20.00 | 20.67 |
| | (comparative | | | | |
| | example) | | | | |
| 2 | 0.50% | 23.00 | 22.00 | 21.50 | 22.50 |
| | (inventive | | | | |
| | example) | | | | |
| 3 | 0.75% | 28.50 | 29.75 | 30.00 | 29.41 |
| | (inventive | | | | |
| | example) | | | | |
| 4 | 1.00% | 23.50 | 21.75 | 23.00 | 22.75 |
| | (inventive | | | | |
| | example) | | | | |

Wherein,

the ejection volume is 0.06 mL/spray;

the potentiating composition is sprayed at 3900 Kpa;

the pH of the solution is 10.30-12.13

the concrete compressive strength of cement is increased by almost 37% using spray potentiator, which leads to reduction of raw material quantity without compromise on quality. For say, instead of 1 kg of cement, 630 gm is sufficient to achieve equal strength as that of 1 kg. As is acknowledged by experts of the field, approximately 10% of building estimate is spent for cement only. So, when 37% of cement consumption is reduced, proportionately water, sand, stones, man power, curing, mixing, cost, time is also reduced. Further, the spray potentiator is combined with other allied products like paint, adhesives, varnish, to increase the strength of iron rods (while melting), bricks, hardening agent, water proofing compound, etc. to increase their quality. FTIR (FIG. 6) and PXRD studies (FIG. 7) also states the other advantageous properties of the present composition i.e. smooth texture and sank rapidly in water, compared to control.

### Example 9: Effect of Spray Potentiator Composition on Electronic Device and Tobacco Products

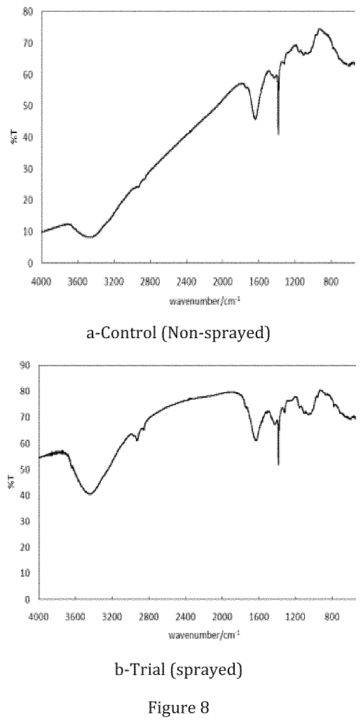

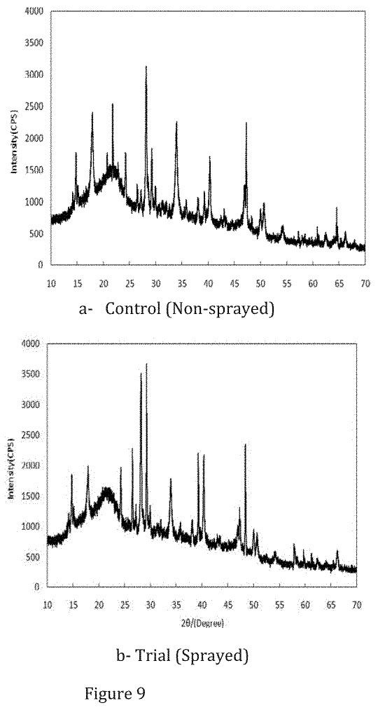

The composition, container and spraying parameter were followed as described in Example 1. The potentiating composition is applied to electric or electronic device and it was found that the radiation effect to health due to electric and electronics equipments is reduced up to 60-90%. Similarly, 20-100% toxins of tobacco, alcohol and allied products are neutralized without compromising on satiety levels of these products, a trial was conducted wherein a universal potentiator solution is sprayed over the laminated cigarette packet and the inside cigarette when smoked and its organoleptic parameters were recorded. The tobacco ground as powder and was then subjected to instrumentation for FTIR and PXRD at Department of Chemistry, University of Tokyo, Tokyo, Japan. It was found that 50% aroma and 60% hotness was reduced in spray form with respect to Control (FIGS. 8 and 9). All the more, the spraying of potentiating composition makes smoke smooth and soft to inhale. Even the smoker's mouth and finger odor vanish in 5-10 minutes using such composition. External spray is enough, and this does not even require tearing off laminated polythene paper over the cigarette packet. It has also been observed that using such a spray, the satiety of smoking remains intact while minimum 75% of nicotine is reduced simultaneously.

With respect to alcohol, the use of this spray potentiator helps to reduce hangover without losing satiety levels. The consumer can thus feel the enhanced taste with a brisk and fresh feeling at same price. This has also reduced shivering and in-coordination among the consumers and even the alcohol excretion from the body is enhanced.

### Example 10: Effect of Spray Potentiator Composition on Agro-Chemicals

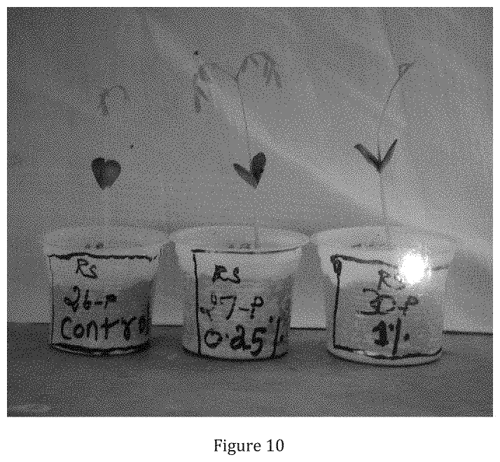

The composition, container and spraying parameter were followed as described in Example 1. In this trial, the material on which the potentiating composition is applied selected from a group consisting of ground net, cow pea and the plant species includes Adenanthera pavonia. In this trial, the spraying composition is sprayed once till the germination phase and twice after germination phase. After the spraying, the growth is increased from 25-50% (FIG. 10).

### Example 11: Comparative Toxicity Studies

In-vitro cytotoxicity of the composition of was carried out on African green monkey kidney cells (Vero cells). The composition was prepared as ‘P’, ‘PFHM’, ‘TPN’ & PFP and the comparative study of these products on the kidney cells was carried out to find out the toxic level of these products considering sodium chloride (NaCl) and sodium hydroxide (NaOH) as Controls:

TABLE 12
Comparative toxicities studies

| | | | | | | | | Non-toxic |
|---|---|---|---|---|---|---|---|---|
| | | | | | | | | level^{#} |
| Composition | Ingredient | | | | | | | (%) |
| P | Sodium | Potassium | Sodium | Sodium | Calcium | Calcium | Magnesium | 0.50% |
| | chloride | nitrate | carbonate | nitrite | carbonate | oxide | oxide | |
| | | | monohydrate | | | | | |
| PFHM | — | Potassium | Sodium | Sodium | Potassium | Sodium | — | 0.50% |
| | | nitrate | carbonate | nitrite | Aluminium | sulphate | | |
| | | | anhydrous | | sulphate | | | |
| TPN | Sodium | Potassium | Sodium | Sodium | — | — | — | 0.12% |
| (Comparative | chloride | nitrate | carbonate | nitrite | | | | |
| example) | | | anhydrous | | | | | |
| PFP | Sodium | Potassium | Sodium | Sodium | — | — | — | 0.25% |
| (Inventive example) | chloride | nitrate | carbonate | carbonate | | | | |
| | | | anhydrous | monohydrate | | | | |
| Control-I | Sodium | — | — | — | — | — | — | 0.50% |
| | chloride | | | | | | | |
| Control-II | Sodium | — | — | — | — | — | — | 0.06% |
| | hydroxide | | | | | | | |

^{#}up to total amount of the composition

According to the toxicity studies as above, the toxic level of the compositions ‘P’, ‘PFHM’ and ‘PFP was found at 0.50% which is corresponding to Control-I (NaCl). On the other hand, the toxic level of TPN (comparative composition) and Control-II (NaOH) was found at lower concentration i.e. 0.12% and 0.06% respectively as compared to inventive composition PFP (0.25%).

The foregoing description is a specific embodiment of the present disclosure. It should be appreciated that this embodiment is described for purpose of illustration only, and that numerous alterations and modifications like Universal Potentiator's powder, liquid, spray and fumes may be practiced by those skilled in the art without departing from the spirit and scope of the invention. It is intended that all such modifications and alterations be included in so far as they come within the scope of the invention as claimed or the equivalents thereof.

We claim:

1. A multipurpose potentiator composition, comprising:
sodium carbonate monohydrate, sodium carbonate, anhydrous, potassium nitrate, sodium chloride and water such that the potentiating composition is applied to alter physical or chemical properties or both of a substance on which the potentiating composition is applied.

2. The multipurpose potentiator composition as claimed in claim 1, wherein the sodium carbonate monohydrate is 35% of the multipurpose potentiator composition.

3. The multipurpose potentiator composition as claimed in claim 1, wherein the sodium carbonate anhydrous is 30% of the multipurpose potentiator composition.

4. The multipurpose potentiator composition as claimed in claim 1, wherein the potassium nitrate is 20% of the multipurpose potentiator composition.

5. The multipurpose potentiator composition as claimed in claim 1, wherein the sodium chloride is 15% of the multipurpose potentiator composition.

6. The multipurpose potentiator composition as claimed in claim 1, wherein the substance is selected from a group consisting of lady finger, pumpkin, carrot, beans, brinjal, bottle guard, bitter guard, jamun, banana, strawberry, mango, papaya, chicken gravy, mutton gravy, raw meat, boiled egg, half boiled egg, omlette, raw egg, raw milk, boiled milk, market raw milk, market boiled milk curd, butter milk, butter, clarified butter, jasmine, rose, chrysanthemum, moringa leaves, spinach, antibiotic, human transfusion blood, food and mouth disease vaccine, petrol, diseal, liquefied petroleum gas, kerosene, gingelly oil, rubber material, cotton yarn, cement, electric and electronic equipments, tobacco products or alcohol.

7. The process for spraying the composition as claimed in claim 1 into the substance comprising the steps of
i) providing a container includes an orifice and a plunger,
ii) placing the composition into the container;
iii) pressing the plunger such that a quantity of the composition is delivered to the substance through an orifice;
wherein diameter of the orifice is 0.3-0.4 mm;
wherein distance between the container and the substance is 6-12 inch;
wherein concentration of the composition is 153 mg/ml;
wherein pressure is 3900 KPa;
wherein quantity of the composition delivered is 60-70 μl.

8. The process as claimed in claim 1, wherein the substance is selected from a group consisting of lady finger, pumpkin, carrot, beans, brinjal, bottle guard, bitter guard, jamun, banana, strawberry, mango, papaya, chicken gravy, mutton gravy, raw meat, boiled egg, half boiled egg, omlette, raw egg, raw milk, boiled milk, market raw milk, market boiled milk curd, butter milk, butter, clarified butter, jasmine, rose, chrysanthemum, moringa leaves, spinach, antibiotic, human transfusion blood, food and mouth disease vaccine, petrol, diseal, liquefied petroleum gas, kerosene, gingelly oil, rubber material, cement, electric and electronic equipments, tobacco products or alcohol.

9. The process for socking a substance into a solution of the composition as claimed in claim 1 comprising the step of socking the substance into the solution for 5-10 minutes.

10. The process as claimed in claim 9, wherein the substance is a cotton yarn.
